(19) United States
(12) Patent Application Publication
(10) Pub. No.: US 2023/0058840 A1
(43) Pub. Date: Feb. 23, 2023

(54) PROCESS FOR RECYCLING LAMINATED POLYMER PACKAGING USING ETHYLENE GLYCOL

(71) Applicants: Edson JOAQUIM ANTUNES QUEVEDO, Barueri (BR); Henrique SILVA DE ARAUJO, Valinhos (BR)

(72) Inventors: Edson JOAQUIM ANTUNES QUEVEDO, Barueri (BR); Henrique SILVA DE ARAUJO, Valinhos (BR)

(21) Appl. No.: 17/785,167

(22) PCT Filed: Nov. 20, 2020
(86) PCT No.: PCT/BR2020/050494

§ 371 Date: Jun. 14, 2022

(30) Foreign Application Priority Data
Dec. 19, 2019 (BR) 10 2019 027314-3

(51) Int. Cl. C08J 11/24

(52) U.S. Cl. CPC C08J 11/24; C08J 2367/02; C08J 2323/06; C08J 2323/12; C08J 2423/06; C08J 2423/12; C08J 2467/02

(57) ABSTRACT

“PROCESS FOR RECYCLING LAMINATED POLYMER PACKAGING USING ETHYLENE GLYCOL” applied in polymeric packaging containing one or more materials from a group formed by PP, PE, PET and aluminum; said process being comprising performing the selective dissolution of PET, reusing it as a product of its reaction with glycol, as well as separating aluminum in its metallic form and PP and PE as a supernatant portion in said product.

### FIELD OF APPLICATION

The present invention refers to the field of application of industrial chemical processes, more precisely in the recycling processes of polymeric packaging with laminated structure 03/06.

The recycling process of the present invention is intended for the recycling of polymeric packaging with a laminated structure and stands out from other recycling processes, as it allows the separation of polymers joined by lamination, which are incompatible with the conventional thermoplastic reprocessing process.

Specifically, the technique achieves the complete separation of PET (polyethylene terephthalate) through its selective dissolution in the medium and the separation of PE (polyethylene) and PP (polypropylene) through its complete melting, in a system of heterogeneous phases, wherein they can be collected directly, thus allowing the recycling of any polymeric package independent of its additives. Said packages can be manufactured in laminated films containing PET in its metallic form or not, aluminum, polyethylene and polypropylene in its metallic form or not. In order to perform the separation, a process comprising ethylene glycol in its mono-, di- or tri-forms is applied, allowing the dissolution of the packaging's polyester films, resulting in elements that can return to the manufacturing process of new packaging, configuring a circular economy.

### DESCRIPTION OF PRIOR ART

Currently, one of the most commented and widespread subjects, both in academia and in industry, concerns the care for the environment, as well as the rational use of natural resources. Thus, concepts such as sustainability, circular economy and recycling are always in the spotlight and in a position to improve.

It is not new that, with the process of global industrialization, there has been an intensification of natural resources use and, consequently, environmental degradation has also escalated, causing the debate on the best use of natural resources and, thus, the reduction of damages caused to the environment, became increasingly urgent.

From this need, much progress has been made regarding the reuse of natural resources and the efficiency of recycling processes of the most diverse products of daily use.

However, some products stand out negatively since their recycling process is difficult, such as multilayer laminated packaging. Such packages do not have effective recycling processes, represent a large part of the domestic waste generated in cities and are also the main pollutants of the oceans.

Undoubtedly, this type of packaging has remarkably interesting characteristics and, consequently, great industrial application, whether as sealing films, food storage and in the packaging of industrial raw materials, among others, so that its use becomes almost essential.

Recycling of this type of laminate packaging is not effective since they are made up of several elements such as, for example, layers of laminate polyethylene terephthalate, both in its metallic form and in its normal form, aluminum, polyethylene, polypropylene in both its metallic form and in its normal form, in addition to any impressions that the packaging may have. Within this variety of packaging elements, the most critical elements are PET and aluminum, since their physical characteristics are quite different in terms of processing and extrusion, when compared to other polymers such as PE or PP, so that the combination of PET and/or aluminum in packaging makes it impossible to reuse PE or PP and vice versa.

Facing this situation, the prior art shows alternatives that try to make the recycling processes of polymeric packaging with laminar structure viable, such as, for example, PI0706115-3, filed on Oct. 22, 2007 intitled “RECICLAGEM DE EMBALAGEM MULTICAMADAS”, owned by UNICAMP. According to its abstract, the patent document refers to a process of separating the materials making up a multilayers package. More specifically, said process allows said multilayers making up plastic, metallic and paper materials to be separated so as not to generate residues harmful to the environment.

Document PI0706115-3, filed on Oct. 22, 2007 intitled “RECICLAGEM DE EMBALAGEM MULTICAMADAS”, shows two steps, the first of which is an immersion bath in an NaOH alkaline medium in order to separate the plastic layer the packaging of the metallic layer; and the second step consisting of a new immersion bath in a NaOH-based alkaline medium, reacting directly with the metallization, resulting in Al³⁺, which is subsequently treated.

The disadvantage of the process showed by document PI0706115-3 lies in the fact the process has only a solution for the metallic layer of the laminate structure package. Therefore, polymeric compounds require further processing, making the process taught by the document ineffective, in addition to not acting in the wide universe of packages that combine PET and PE or PP without having at the joining interface a metallic layer or aluminum film.

Document BR102013023494-0, filed on Sep. 13, 2013 intitled “PROCESSO DE SEPARAÇÃO E RECICLAGEM QUÍMICA DE EMBALAGENS MULTICAMADAS”, shows, according to its abstract, an alternative for the recycling of multilayer packaging containing PET (Polyethylene terephthalate), aluminum and PE (Polyethylene) by chemical recycling of this type of packaging through the delamination of polymers and the depolymerization of PET using hydrolysis reaction.

Document BR102013023494-0 introduces the use of an alcohol for recycling the laminate packaging, however, the product of the process will still result in PE and PET together so that the extrusion of the polymers will be unfeasible given the difference in density of both.

Some other solutions are showed in other patent documents such as US2009011213 and WO2018025058 which share the same difficulties, that is, they have processes based on a NaOH alkaline bath, which attacks the aluminum metallization layer through selective dissolution, however do not allow the reuse of polymers such as PE and PP, or PET, whenever there is PET directly bonded to PE or PP without aluminum interleaved in the bonding interface.

Therefore, the prior art would benefit from a process that allows the complete separation of PET through its selective dissolution in ethylene glycol, as well as the separation of PE and PP films through their complete melting in the medium, generating a system of heterogeneous phases, in which PP and PE can be collected directly on the medium surface and wherein aluminum, if present, can be collected in its metallic form through the formation of a precipitate (decantation) so that the PET and the other mentioned components can be separated and reused.

### SUMMARY OF INVENTION

The present invention aims to show a recycling process applied to polymeric packaging with a laminate structure, which allows the separation and reuse in a circular economy of its respective layers, regardless of the way or sequence in which they are combined.

Another objective of the invention is to show a process allowing the aluminum layer to be reused in its metallic form, and not as salts.

It is also an objective of the present invention to show a process wherein the bath is used as a raw material in the production of PET, thus configuring a circular economy.

### BRIEF DESCRIPTION OF THE INVENTION

The invention achieves the described objectives from the use of an ethylene glycol-based bath, being able to use its forms: mono-, di- or tri- instead of an NaOH alkaline bath.

Considering that ethylene glycol is a manufacture precursor of polyethylene terephthalate (PET), the product of the selective dissolution of PET in mono-ethylene glycol can be directly reintroduced in the polycondensation step in the PET manufacture, serving as raw material for this process.

PET final form after bathing allows its reuse through chemical recycling into the production of new PET polymers, thus configuring a circular economy with minimal material loss.

The ethylene glycol bath also allows an effective separation of the aluminum layer in a reaction resulting in metallic aluminum, which has greater economic value compared to aluminum salts obtained in a NaOH bath.

Finally, the ethylene glycol reaction itself results in a compound that can return to PET production, requiring only enrichment with terephthalic acid in the polycondensation step, so that the reuse of materials is almost complete.

### BRIEF DESCRIPTION OF THE DRAWINGS

The subject matter of this Invention will be fully clear in its technical aspects from the detailed description that will be made based on the FIGURES listed below, wherein:

FIG. 1 shows a block diagram referring to the steps and sub-steps comprised by the polymeric packaging recycling process through ethylene glycol, referring to the correlation between them.

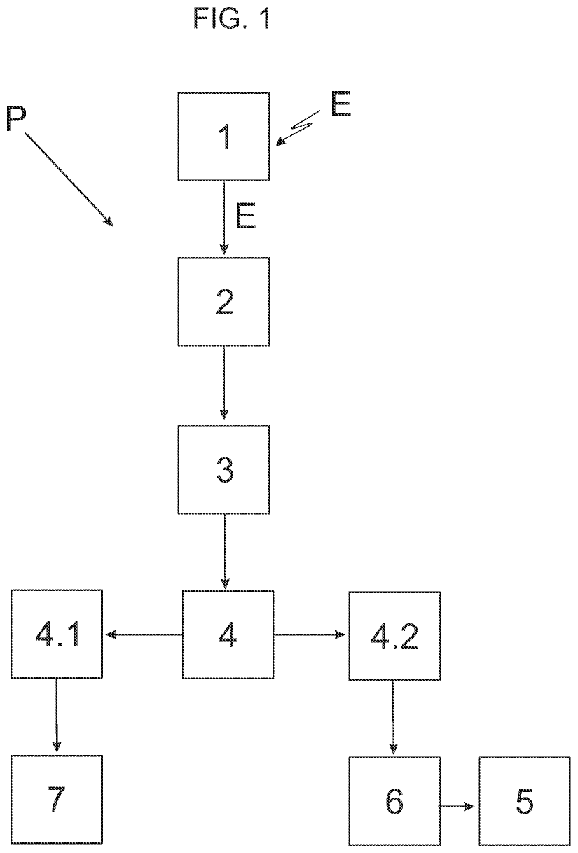

### DETAILED DESCRIPTION OF THE INVENTION

In accordance with the objectives showed through the brief description, the present patent application “PROCESS FOR RECYCLING LAMINATED POLYMER PACKAGING USING ETHYLENE GLYCOL” shows a process (P) aiming at the recycling of polymeric packaging (E) endowed with a laminar structure comprising PET, PE, PP and/or aluminum, regardless the amount or combination between them.

In this process (P), PET is separated through a chemical reaction, while aluminum, PE and PP are separated through physical processes. In detail, PET is chemically separated through its complete selective dissolution, which takes place through a reaction with glycol, which can be treated as: glycolysis, transesterification or alcoholysis in the literature. PE and PP are physically separated when they reach a temperature above their melting points, then undergo melting and, as they form a heterogeneous system with glycol, float in the solution. Aluminum remains solid, without the presence of any adhesive polymer. Finally, the metallic aluminum precipitates and settles to the bottom of the bath.

The process (P) has the following consecutive steps:

(i) granulation (1) of the polymeric package (E);

(ii) immersion (2) of the polymeric package (E) in a glycol bath;

(iii) selective dissolution (3) of the polymeric package (E);

(iv) separation (4) of polymers;

(v) washing (5) the polymers;

(vi) griding (6) the polymers;

(vii) separation of aluminum (7) after the selective dissolution step (3); and

(viii) cleaning and drying (8) of the aluminum.

The granulation step (1) of the polymeric package (E) consists of fragmenting the package into small portions, aiming at the smallest possible particle size, in order to accelerate the PET selective dissolution step (3).

The granulation step (1) is performed with the aid of industrial equipment capable of cutting and grinding the packages (E). Said industrial equipment also performs the cleaning of said packages (E) by passing them after cutting and grinding into a gutter with water. After the packages (E) have passed through the gutter with water, they are dried by the industrial equipment itself through a centrifuge and air blowing process.

The immersion step (2) takes place after the granulation step (1), so that the packages (E) are cut. This immersion step (2) is performed in a chemical reactor, preferably made of stainless steel; and in an inert atmosphere with N₂, wherein the aforementioned chemical reactor has an upper inlet for nitrogen purge at a flow between five to ten L/min.

Still in the immersion step (2), the packages (E) are immersed in a pure glycol solution, preferably in mono-ethylene glycol, which can be performed in di-ethylene glycol or tri-ethylene glycol, however, not being limited to these types of glycols. Pure glycol comprises molecular structure I shown below:

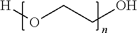

wherein, brackets “[ ]” represent a single glycol unit and “n” is the index referring to the number of glycol units for a given pure glycol used in the process (P). For the purposes of the present invention, the value of “n” comprises the range between 1 and 10, preferably n=1, n=2 or n=3, however, not being limited to these values. When n=1, n=2 and n=3 the resulting pure glycol is, respectively, mono-ethylene glycol, di-ethylene glycol and tri-ethylene glycol.

The immersion step (2) may comprise heating and stirring sub-steps, so that in the heating sub-step the glycol is heated by means of a thermal fluid heater, heating the reactor via a coil, raising the temperature of the glycol to a range between 180° C. and 240° C., with respect to the glycol boiling temperature used, that is, 197.6° C. for mono-ethylene glycol, 245° C. for di-ethylene glycol and 285° C. for tri-ethylene glycol. A top condenser can be installed to recover steams from the glycol, increasing process throughput.

At stirring sub-step of the immersion stage (2), the chemical reactor is stirred through a system comprising a motor and a geared motor, so that the packages (E) are uniformly mixed with the glycol, thus facilitating the PET selective dissolution. As the immersion step (2) is performed in a closed vessel condition, the volume of packages (E) must respect the range between 40 to 60% of the glycol volume, thus facilitating the separation of the supernatants PE and PP and precipitated aluminum.

The selective dissolution step (3) of the package (E) starts after the stabilization of the glycol temperature at the immersion step (2). Said selective dissolution step (3) can be accelerated, that is, with the glycol at the appropriate temperature, the immersed packages (E) are accelerated by means of Cowles-type propellers, circulation and/or pumping gears or circulation through screens metallic, so that acceleration contributes to the shearing of the supernatant material composed of PE or PP that can trap particles to be separated.

The selective dissolution step (3) time will depend on the size of the packages (E), more precisely on the size of the portions achieved in the granulation step (1), ranging from 20 to 60 minutes. A visual indicator of the selective dissolution step (3) end is that it will not be possible to visualize films suspended in the glycol, with only a kind of supernatant sludge corresponding to PP and PE being visible. At the selective dissolution step (3) end, the result of the glycol and the package (E) reaction will be a suspension of PP and PE, or one of the two, depending on the initial configuration of the package; aluminum precipitated in its metallic form, if the package (E) contains aluminum; and at least one type of terephthalic acid-based polyol.

The chemical reaction between glycol and PET, called glycolysis, transesterification or alcoholysis, is represented below for the purpose of better understanding of the present invention, however, not being limited to this type of reaction. In this non-restrictive example, the glycol, of molecular structure I, chemically reacts with PET, of molecular structure II, to form a first type of terephthalic acid-based polyol of molecular structure III, being a by-product of the reaction. In this reaction, regardless of the glycol type used for the reaction with PET, another by-product formed will be mono-ethylene glycol, of molecular structure IV, as this by-product comes from the mono-ethylene units present in PET. The molecular structure II of PET comprises “m” terephthalate units represented in brackets “[ ],” so that “m” is equal to or greater than 1. Said molecular structure III polyol comprises “x” terephthalate units represented in brackets “[ ],” so that “x” is equal to or greater than 1 and has values lower than “m”. In this context, “m” will always be greater than “x” (m>x). Still on said polyol of molecular structure III, it can indefinitely undergo further reactions with the mono-ethylene glycol by-product of molecular structure IV or with the glycol of molecular structure I, in case of excess of said glycol in the medium, resulting in a second type of terephthalic acid-based polyol of molecular structure V, a product of the complete reaction. This second type of polyol comprises the bis(2-hydroxyalkyl) terephthalate product, however, not being limited to that second type of polyol. Although complete reaction between PET and glycol is desired, at least two types of polyol can be simultaneously present. In a reaction between PET and glycol comprising values of n=1 (mono-ethylene glycol), 2 (di-ethylene glycol), 3 (tri-ethylene glycol), 4 (tetra-ethylene glycol), 5 (penta-ethylene glycol), 6 (hexa-ethylene glycol), 7 (hepta-ethylene glycol), 8 (octa-ethylene glycol), 9 (nona-ethylene glycol) or 10 (deca-ethylene glycol), the alkyl function of the second type of polyol formed bis(2-hydroxyalkyl) will be selected, respectively, from the group comprising ethylene, di-ethylene, tri-ethylene, tetra-ethylene, penta-ethylene, hexa-ethylene, hepta-ethylene, octa-ethylene, nona-ethylene or deca-ethylene, however, not being limited to said values of “n” and the possible bis(2-hydroxyalkyl) products mentioned. Among numerous possibilities of the second type of polyol, the products bis(2-hydroxyethylene), bis(2-hydroxydi-ethylene) and bis(2-hydroxytri-ethylene) are preferred when the glycol comprises the values of n=1, 2 or 3, respectively.

In the selective dissolution step (3), the glycol reacts in the packaging, specifically attacking the PET, so that the PET is dissolved, releasing the aluminum that precipitates in the glycol by density difference, while the PET reacts with the glycol forming at least one polyol. The PP and PE portions, on the other hand, melt due to the high temperature of the medium, but remain insoluble in the glycol, thus rising to the surface of the liquid, also due to the difference in density.

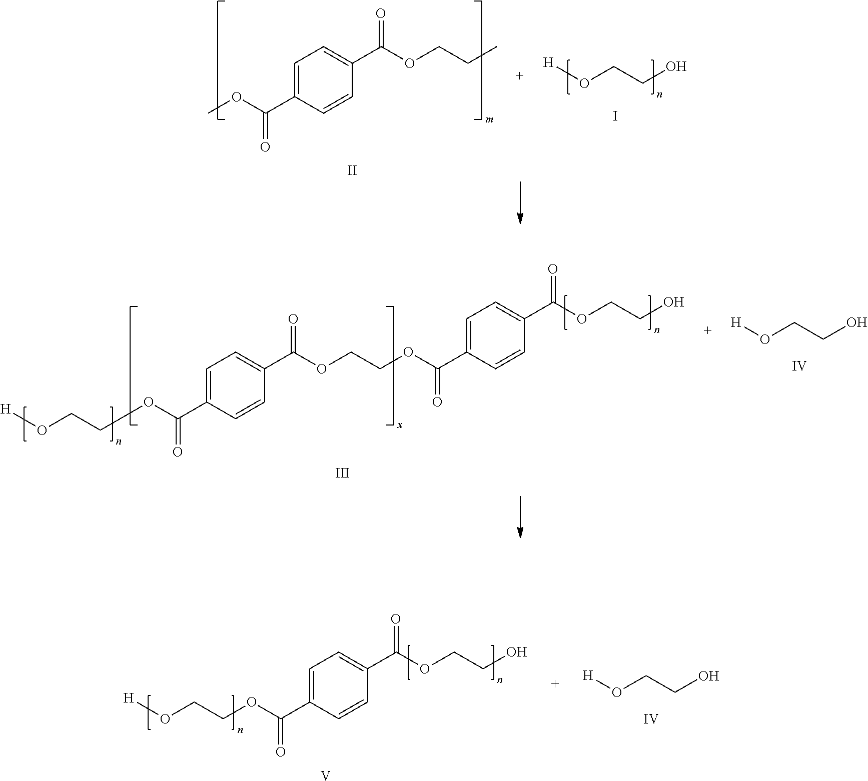

The separation step (4) occurs after the selective dissolution step (3) and complete melting of the PP and/or PE films. Said separation step (4) comprises the sub-steps of aluminum separation (4.1) and PE/PP separation (4.2).

The aluminum separation sub-step (4.1) consists of draining a small fraction of the bath from the bottom of the reactor, while hot, followed by passing the liquid from this bath through a filtration line, so that the aluminum that has already precipitated is retained and the glycol returned to the reactor. In order to facilitate the aluminum separation step (4.1), the reactor preferably has a conical or torispherical bottom, thus helping to remove the aluminum precipitated by the reactor bottom valve. In turn, the filtration line has a display through which it is possible to visualize the amount of aluminum retained, since the aluminum separation step (4.1) will only be completed when all the precipitated aluminum is retained in the filter line.

The PE/PP separation sub-step (4.2) consists of draining the bath and the supernatant portion through pumping, by means of side drains located in the reactor, directing them to a fine metallic screen filter, in order to remove or retain any unwanted particles which are adhered to the supernatant portion. After removing unwanted particles, the mixture between bath and supernatant portion is cooled so that the supernatant portion solidifies and is then physically removed, while the liquid from the bath is pumped back to the reactor.

The washing step (5) of the polymers takes place after the separation step (4) and, is performed through water to remove the glycol residues present both in PE and PP in an equipment similar to that used in the step of granulation (1). Thus, polymers are subjected to a grinding step (6), before the washing step (5) and then they go to a gutter with water, configuring the washing step (5), where all glycol residues are removed and, after decontamination, the polymers already cleaned are dried. Given the low solubility of glycol in PE or PP, the washing step (5) is highly effective, completely minimizing glycol particles.

The polymer grinding step (6) is performed before the washing step (5) in order to increase the contact area of the material with the water in the washing step (5).

The aluminum cleaning and drying step (7) takes place after the aluminum separation sub-step (4.1) and has the purpose of removing the glycol particles in it, preparing the material for later use. Said cleaning and drying step (7) of the aluminum is performed through water curtain-type direct rinsing on a conveyor or into a gutter.

The polyols resulting from the selective dissolution of PET can be reused in two ways, the first being as a reagent in consecutive processes, returning to the reactor as a bath. However, the use of polyols in consecutive processes gradually increases its viscosity, due to the reaction between PET and the polyols itself, so that their consecutive use will only be possible as long as their viscosity allows.

The second way is when the glycol already has a viscosity that prevents its use in consecutive processes, being subjected to a re-polymerization process through the addition of terephthalic acid in the PET polycondensation. To know the amount of terephthalic acid needed for the re-polymerization process, it is essential to measure the number of free hydroxyl of the polyols obtained (mgKOH/g).

The great advantage of the process (P) compared to other polymeric packaging (E) recycling processes with a laminate structure is to show an economically viable and effective way to reuse the PET present in this type of polymeric package (E). Not to mention that this process manages to completely remove PET from other materials, and this is a great advantage, since the presence of PET waste creates important technical problems in the recovery of PE and PP, as it has quite different melting characteristics and this is the main practical reason recycling of multi-layer laminates is currently unfeasible.

Another particularly important advantage of the aforementioned process (P) lies in the fact that it configures a circular economy, that is, all products from the process (P) can be re-used in the polymeric packaging (E) production chains themselves.

It should be understood the present description does not limit application to the details described herein and that the invention is capable of other embodiments and being practiced or performed in a variety of ways, within the scope of the claims. Although specific terms have been used, such terms should be interpreted in a generic and descriptive sense and not for the purpose of limitation.

1. A PROCESS FOR RECYCLING LAMINATED POLYMER PACKAGING USING ETHYLENE GLYCOL having a recycling process for polymeric packaging, characterized in that the process (P) is applied to polymeric packaging (E) comprising one or more materials from a group formed by PET, PE, PP and aluminum, with the consecutive steps:
(i) granulation (1) of the polymeric package (E);
(ii) immersion (2) of the polymeric package (E) in a glycol bath;
(iii) selective dissolution (3) of the polymeric package (E);
(iv) separation (4) of polymers, comprising the sub-steps of aluminum separation (4.1) and PE/PP separation (4.1);
(v) washing (5) of the polymers after the separation step (4), removing the glycol residues present in both PE and PP;
(vi) grinding (6) the polymers before the washing step (5);
(vii) cleaning and drying (7) the aluminum after the aluminum separation sub-step (4.1);
the washing (5) and grinding (6) steps are performed in an industrial equipment capable of cutting and grinding the packages (E), having gutters with water and a centrifuge and air blowing drying system.

2. The PROCESS, according to claim 1, characterized in that the glycol comprises molecular structure I and PET comprises molecular structure II: 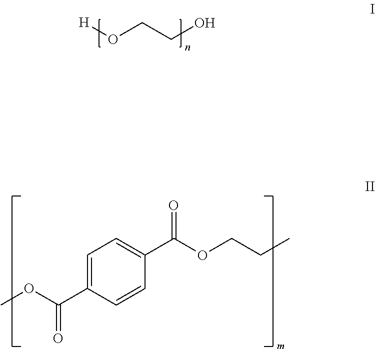
wherein:
“n” is equal to values comprised in the range between 1 and 10, preferably between 1 and 3; and
“m” is equal to or greater than 1.

3. The PROCESS, according to claim 1, characterized in that the granulation step (1) fragments the polymeric package (E) into small portions in an industrial equipment capable of cutting and grinding the packages (E), having gutters with water and centrifuge and air blow drying system.

4. The PROCESS, according to claim 1, characterized in that the immersion step (2) is performed after the granulation step (1) in a chemical reactor preferably built of stainless steel and in a controlled atmosphere with N₂, and the chemical reactor having an upper inlet for nitrogen purge at a flow between five to ten L/min; and a conical or torispherical bottom.

5. The PROCESS, according to claim 4, characterized in that the immersion step (2) is performed in a pure glycol bath, in a closed vessel condition, and the volume of packages (E) must respect the range between 40 to 60% glycol volume.

6. The PROCESS, according to claim 4, characterized in that the immersion step (2) may comprise heating and stirring sub-steps, wherein the stirring sub-step takes place through a system comprising motor and geared motor.

7. The PROCESS, according to claim 6, characterized in that the heating sub-step is performed through a thermal fluid heater containing coil, raising the glycol temperature to a range between 180° C. and 240° C.

8. The PROCESS, according to claim 1, characterized in that the selective dissolution step (3) is performed after the glycol temperature stabilization in the immersion step (2) lasting between twenty and sixty minutes.

9. The PROCESS, according to claim 8, characterized in that the selective dissolution step (3) can be accelerated through Cowles-type propellers, circulation and/or pumping of gears or circulation through metallic screens.

10. The PROCESS, according to claim 8, characterized in that the product of the selective dissolution step (3) will be a suspension of PP and PE; aluminum precipitated in its metallic form; and at least one terephthalic acid-based polyol comprising molecular structure III or molecular structure V: 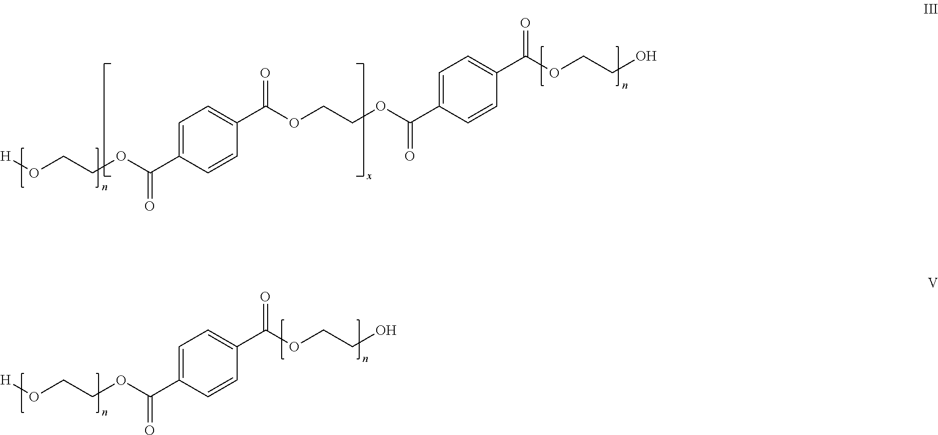
wherein:
“n” is equal to values comprised in range between 1 and 10, preferably between 1 and 3;
“x” is equal or greater than 1;

11. The PROCESS, according to claim 1, characterized in that the separation step (4) takes place after the selective dissolution step (3) and complete melting of the PP and/or PE films, and the separation step (4) comprising the aluminum separation (4.1) and PE/PP separation (4.2) sub-steps.

12. The PROCESS, according to claim 11, characterized in that the aluminum separation sub-step (4.1) consists of draining a small fraction of the bath from the reactor bottom, under heat, followed by passing this bath through a filtration line.

13. The PROCESS, according to claim 11, characterized in that the PP/PE separation sub-step (4.2) consists of draining the bath and the supernatant portion through pumping, through side drains located in the reactor, directing them to a fine metallic screen filter, cooling the supernatant portion and returning the bath to the reactor by pumping.

14. The PROCESS, according to claim 1, characterized in that the process (P) can use mono-ethylene glycol, di-ethylene glycol and tri-ethylene glycol as glycol.

15. The PROCESS, according to claim 1, characterized in that the aluminum cleaning and drying step (7) takes place after the aluminum separation sub-step (4.1), removing the glycol particles at the aluminum through water curtain-type direct washing, on a conveyor or gutter.
